(19) United States
(12) Patent Application Publication
(10) Pub. No.: US 2024/0203573 A1
(43) Pub. Date: Jun. 20, 2024

(54) DATA PROCESSING SYSTEM AND METHOD THEREOF

(71) Applicant: INDUSTRIAL TECHNOLOGY RESEARCH INSTITUTE, Hsinchu (TW)

(72) Inventors: Yue-Min JIANG, New Taipei City (TW); Jian-Ren CHEN, Hsinchu City (TW); Min-Yi HSIEH, Taipei City (TW)

(73) Assignee: INDUSTRIAL TECHNOLOGY RESEARCH INSTITUTE, Hsinchu (TW)

(21) Appl. No.: 18/080,855

(22) Filed: Dec. 14, 2022

(51) Int. Cl. G16H 40/40; G06F 16/31; G16H 20/00; G16H 50/70

(52) U.S. Cl. CPC G16H 40/40; G06F 16/313; G16H 20/00; G16H 50/70

(57) ABSTRACT

A data processing method includes the following steps. According to a wound symptom record, a trend of each wound state is analyzed. According to product data of the medical appliances in the treatment record, featured text information are extracted. First correlation weight values and second correlation weight values of the wound states are generated, the first correlation weight values indicate correlations between the wound states and the medical appliances, and the second correlation weight values indicate correlations between the wound states and the featured text information. A query condition is generated according to a target state, and a target nursing keyword is generated according to the query condition. Recommended medical appliances are selected from the medical appliances according to the target nursing keyword and the second correlation weight values.

### TECHNICAL FIELD

The present disclosure relates to a data processing system and a method thereof, in particular relates to a data processing system and a method thereof for recommending medical appliances according to wound state.

### BACKGROUND

Chronic wounds heal slowly, resulting in huge healthcare expenditures. The nursing of complex wounds relies more on professional experience. Compared with general nurses, professional wound care practitioner may handle complex wounds, but those having professional wound care practitioner licenses are insufficient.

In the treatment of wounds, as the degree of healing of the wounds is different, the medical appliances that need to be provided are also different. Taking medical appliances as “dressings” as an example, the number of items of dressings is huge (for example, a professional wound care practitioner has more than 3,000 items of dressings), and the amount of information of the scientific name, product name, usage scheme and precautions of the dressings, etc. is huge. It is difficult to accurately select appropriate dressings to treat the wound.

In response to the above technical problems, an intelligent data processing system may be used to select and recommend medical appliances (such as dressings), which may greatly reduce the cost of wound care. Therefore, those skilled in the art devote to develop a data processing system and a data processing method, which recommends the most suitable medical appliances for wound states.

### SUMMARY

According to an aspect of the present disclosure, a data processing system is provided. The data processing system includes the following elements. A first database, for storing a wound symptom record and a treatment record, the wound symptom record comprises a plurality of wound states, and the treatment record comprises a plurality of medical appliances corresponding to the wound states. A second database, for storing a plurality of product data of the medical appliances. A first processing engine, for analyzing the first database to analyze a trend of each of the wound states according to the wound symptom record, and analyzing the second database to extract a plurality of featured text information from the product data. A second processing engine, for generating a plurality of first correlation weight values and a plurality of second correlation weight values of the wound states, the first correlation weight values indicate correlations between the wound states and the medical appliances, the second correlation weight values indicate correlations between the wound states and the featured text information. A third processing engine, for generating a query condition according to a target state, generating a target nursing keyword according to the query condition, and selecting a plurality of recommended medical appliances from the medical appliances according to the target nursing keyword and the second correlation weight values. Wherein, the query condition is related to the trend of each of the wound states, and the target nursing keyword is related to the target state.

According to another aspect of the present disclosure, a data processing method is provided. The data processing method includes the following steps. Storing a wound symptom record and a treatment record in a first database, the wound symptom record comprises a plurality of wound states, and the treatment record comprises a plurality of medical appliances corresponding to the wound states. Storing a plurality of product data of the medical appliances in a second database. Analyzing the first database to analyze a trend of each of the wound states according to the wound symptom record and analyzing the second database to extract a plurality of featured text information from the product data, through a first processing engine. Generating a plurality of first correlation weight values and a plurality of second correlation weight values of the wound states through a second processing engine, the first correlation weight values indicate correlations between the wound states and the medical appliances, the second correlation weight values indicate correlations between the wound states and the featured text information. Generating a query condition according to a target state, generating a target nursing keyword according to the query condition, and selecting a plurality of recommended medical appliances from the medical appliances according to the target nursing keyword and the second correlation weight values, through a third processing engine. Wherein, the query condition is related to the trend of each of the wound states, and the target nursing keyword is related to the target state.

### BRIEF DESCRIPTION OF THE DRAWINGS

FIG. 1 is a block diagram of a data processing system according to an embodiment of the present disclosure.

FIG. 2 is a flow chart of adaptive updating for the lexicon of the “medical appliance keyword”, which is performed by the first processing engine.

FIGS. 3A and 3B are flowcharts of a data processing method according to an embodiment of the present disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically illustrated in order to simplify the drawing.

### DETAILED DESCRIPTION

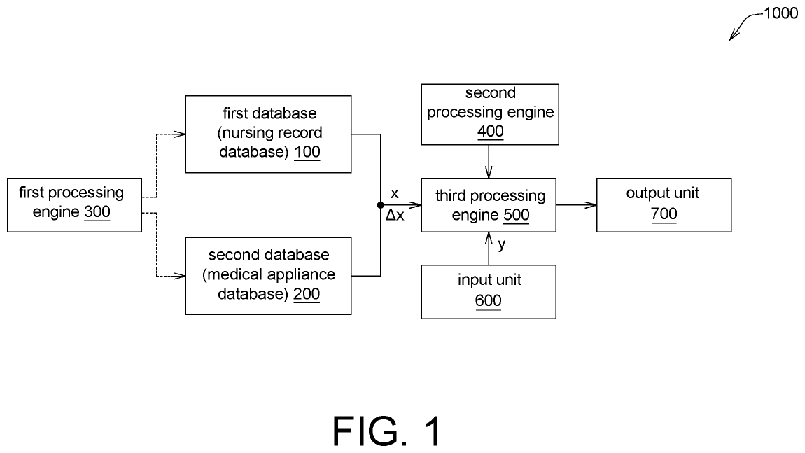

Please refer to FIG. 1, which shows a block diagram of a data processing system 1000 according to an embodiment of the present disclosure. The data processing system 1000 includes a first database 100, a second database 200, a first processing engine 300, a second processing engine 400, a third processing engine 500, an input unit 600 and an output unit 700.

The user of the data processing system 1000 may be a nursing staff. The nursing staff analyzes the symptom state of the affected part (e.g., traumatic wound) of the current case (e.g., a patient) through the data processing system 1000, and the nursing staff selects the desired target state y. Moreover, the data processing system 1000 stores the wound symptom records and treatment records of all historical cases, so as to analyze the wound state x and its state trend Δx of the cases, and analyze the product data of all medical appliances. Based on the above analysis, the data processing system 1000 selects the “recommended medical appliances” which are most suitable for treating the affected part of the current case, from all the medical appliances. Medical appliances are, for example, dressings, which are applied to wounds.

The data processing system 1000 is executed on a system platform of a server host, a personal computer or a mobile computing device. The first database 100 and the second database 200 are, for example, storages (such as memory or hard disk) inside the data processing system 1000. The first database 100 and the second database 200 may also be remote storage spaces outside the data processing system 1000 or Internet databases.

The first processing engine 300, the second processing engine 400 and the third processing engine 500 are, for example, three independent processors inside the data processing system 1000, or, the first processing engine 300, the second processing engine 400 and the third processing engine 500 may be integrated in a same processor. The first processing engine 300, the second processing engine 400 and the third processing engine 500 may be executed according to artificial intelligence algorithms and data models.

The input unit 600 and the output unit 700 are man-machine-interfaces (MMIs), such as graphics-user-interfaces (GUIs). A user (nursing staff) inputs a desired target state y through the input unit 600. In addition, the user obtains a product name of the “recommended medical appliances” through the output unit 700.

More specifically, in this embodiment, the first database 100 is a “nursing record database”. The contents stored in the first database 100 comprise: records of wound symptoms of all cases (hereinafter referred to as “wound symptom record”), and the treatment schemes performed by nursing staff on the wounds of the above cases (hereinafter referred to as “treatment record”).

Table 1-1 is an example of “wound symptom record” stored in the first database 100. Table 1-1 includes the wound symptom record of three cases A001˜A003. The wounds of cases A001˜A003 are, for example, arm epiderm cuts. The wound symptoms or the corresponding wound states comprise: the size of the wound, the growth degree of the wound granulation, the degree of necrosis of the wound, the exudation amount of the tissue fluid exuded from the wound, and the color of the exuded tissue fluid, etc.

Take the case A001 in Table 1-1 as an example. The wound of case A001 is an arm epiderm cut. The size of the wound was 10 mm×5 mm. The growth area of wound granulation accounted for 10% of the entire wound area, and the area of necrotic part of the wound accounted for 45% of the entire wound area. In addition, the exudation amount of tissue fluid exuded from the wound was 25%, and the color of the exuded tissue fluid is yellow.

TABLE 1-1
Wound symptom record (Wound symptom,
or corresponding wound state)

| | | size | | | | |
|---|---|---|---|---|---|---|
| | | (length × | | | | |
| | | width) | | | Tissue | |
| case | wound | (unit: mm) | granulation | Necrosis | fluid | color |
| A001 | Arm | 10 × 5 | 10% | 45% | 25% | yellow |
| | epiderm | | | | | |
| | cuts | | | | | |
| A002 | Arm | 15 × 8 | 50% | 20% | 50% | pink |
| | epiderm | | | | | |
| | cuts | | | | | |
| A003 | Arm | 30 × 20 | 30% | 50% | 25% | pink |
| | epiderm | | | | | |
| | cuts | | | | | |

On the other hand, Table 1-2 is an example of “treatment record” stored in the first database 100. Table 1-2 comprises the treatment schemes (that is, the nursing methods) performed by the nursing staff on the wounds of the three cases A001˜A003. The treatment schemes are, for example, applying medical appliances to the wounds of the three cases A001˜A003. The medical appliances are, e.g., dressings.

The treatment records in Table 1-2 correspond to the wound symptom records in Table 1-1. In the treatment records in Table 1-2, the nursing staff applied dressing D01 to the wound of case A001. The dressing D01 has a product name a1 (that is, a product name of medical appliance). The product name a1 of the dressing D01 is, for example, “skin affinity gel of “A brand””. The size of dressing D01 is 10 mm×10 mm, which corresponds to the wound size with 10 mm×5 mm of the case A001. The cost (i.e., price) of the dressing D01 is 300 dollar per piece. And, the usage frequency of dressing D01 applied to case A001 is 2 pieces per day (i.e., changed 2 times per day).

Similarly, the treatment records in Table 1-2 also comprise the records of applying medical appliances to the wounds of case A002 and case A003. The dressing D02 is applied to case A002, and the product name a2 of the dressing D02 is, for example, “wound powder of “B brand””. Dressing D03 is applied to case A003, and the product name a3 of dressing D03 is, for example, “non-stick wound dressing of “C brand””.

TABLE 1-2
Treatment record

| | | | Size | | Usage |
|---|---|---|---|---|---|
| | Medical | | (length × | Cost(price) | frequency |
| | appliance | Product | width) | (unit: | (unit: |
| Case | (dressing) | name | (unit: mm) | dollar) | times/day) |
| A001 | D01 | a1 | 10 × 10 | 300 | 2 |
| A002 | D02 | a2 | 15 × 15 | 450 | 2 |
| A003 | D03 | a3 | 30 × 30 | 1000 | 4 |

In operation, the first processing engine 300 analyzes and processes the wound symptom records and treatment records stored in the first database 100. For example, from the wound symptom record in the first database 100, the first processing engine 300 fetches the field corresponding to the “wound state x” corresponding to the wound symptom, and the first processing engine 300 performs normalization (or referred to as “standardization”) for each value of wound state x, so as to obtain the normalized value of the wound state x shown in Table 1-3. The normalized values of each item range from 0 to 1.

TABLE 1-3
Normalized values of wound state x

| | | Size | | | | |
|---|---|---|---|---|---|---|
| | | (length × | | | | |
| | | width) | | | Tissue | |
| case | wound | (unit: mm) | granulation | Necrosis | fluid | color |
| A001 | Arm | 0.2 × 0.1 | 0.1 | 0.45 | 0.25 | 0.2 |
| | epiderm | | | | | |
| | cuts | | | | | |
| A002 | Arm | 0.3 × 0.16 | 0.5 | 0.2 | 0.5 | 0.4 |
| | epiderm | | | | | |
| | cuts | | | | | |
| A003 | Arm | 0.6 × 0.4 | 0.3 | 0.5 | 0.25 | 0.4 |
| | epiderm | | | | | |
| | cuts | | | | | |

On the other hand, from the treatment record of the first database 100, the first processing engine 300 fetches the field corresponding to the treatment scheme of the wound symptom, and normalizes each value of the treatment record to obtain normalized values of the treatment scheme shown in Table 1-4, with a range of 0 to 1.

TABLE 1-4
Treatment record

| | | | Size | | Usage |
|---|---|---|---|---|---|
| | Medical | | (length × | Cost(price) | frequency |
| | appliance | Product | width) | (unit: | (unit: |
| Case | (dressing) | name | (unit: mm) | dollar) | times/day) |
| A001 | D01 | a1 | 0.2 × 0.2 | 0.2 | 0.1 |
| A002 | D02 | a2 | 0.3 × 0.3 | 0.3 | 0.1 |
| A003 | D03 | a3 | 0.6 × 0.6 | 0.66 | 0.2 |

Moreover, the first processing engine 300 makes statistics on the wound symptom records stored in the first database 100 to obtain the time-varying trend Δx of the wound state x of the same case. Table 1-5 shows that, the state x(n) of various wound symptoms of case A001 at time points t(n=0)˜t(n=3). According to the wound state x(n) in Table 1-5, the first processing engine 300 calculates the state trend Δx of the wound state x(n) at the time points t(n=0)˜t(n=3). The state trend Δx is defined as: the difference between the wound state x(n) at the current time point t(n) and the wound state x(0) at the first time point t(n=0) divided by the wound state x(0) at the first time point t(n=0), and then divided by the difference period between the first time point t(n=0) and the current time point t(n) (i.e., the period between visits of case A001). Then, being normalized according to 100%, as shown in formula (1):

$$\frac{wound state x ( n ) - wound state x ( 0 )}{wound state x ( 0 ) \times [ t ( n ) - t ( n = 0 ) ]} \times 100 \% \qquad formula ( 1 )$$

The state trend Δx(2) of the granulation growth area of the wound of case A001 at time point t(n=2) is calculated as formula (2):

$$Δ x ( 2 ) = \frac{15 - 40}{40 \times [ 60 days ]} \times 100 \% = - 1.04 \% \qquad formula ( 2 )$$

TABLE 1-5
wound state x(n) of each time point

| | | Time | | | | Wound |
|---|---|---|---|---|---|---|
| Case | wound | point | granulation | Necrosis | Carrion | shrinking |
| A001 | Arm | t(n = 0) | 40% | 65% | 35% | 85% |
| | epiderm | Jan. | | | | |
| | cuts | t(n = 1) | 30% | 45% | 20% | 75% |
| | | Feb. | | | | |
| | | t(n = 2) | 15% | 25% | 15% | 60% |
| | | Mar. | | | | |
| | | t(n = 3) | 10% | 5% | 5% | 45% |
| | | Apr. | | | | |

Please refer to FIG. 1 again, in the data processing system 1000 of the present disclosure, the second database 200 is a “medical appliances database”, and the second database 200 stores various product data of medical appliances. Taking the dressing D03 as an example for the medical appliance, the second database 200 stores: the license number of the dressing D03, the product name a3 of the dressing D03, the manufacturer for the dressing D03, the grade of the medical equipment, the product description, and so on. The above product data of medical appliances may be obtained through analysis of the “medical equipment identification system information management platform”, the “medical equipment license case database” outside the data processing system 1000, or the instruction sheets of dressing D03.

Table 2-1 shows: various product data of the dressing D03 stored in the second database 200. The product data shown in Table 2-1 may be obtained from the analysis of instruction sheet of dressing D03. The product name a3 of dressing D03 is: Non-stick wound dressing of “C brand”. The indications of dressing D03 are: wounds of cosmetic surgery, post-caesarean section, general trauma, and initial pressure sores. The product features of the dressing D03 are: sterilization, absorbing wound exudate, thin design to facilitate wound observation, no sticking to the wound when the dressing is removed, polyurethane film may prevent allergies, and prevent moisture intrusion to reduce infection. The ingredients of dressing D03 is: polyurethane foam, polyurethane film, pressure-sensitive adhesive and PP carving paper.

TABLE 2-1
Product data of dressing D03 (analyzed
from instruction sheet of dressing D03)

| Product | |
|---|---|
| name | |
| a3 | Non-stick wound dressing of “C brand” |
| Indications | Cosmetic surgery |
| | After caesarean section |
| | General trauma |
| | Primary pressure ulcer |
| Product | Sterilization |
| feature | Absorbs exudate from wounds |
| | Thin design for easy wound observation |
| | Not stick to the wound when removing the dressing |
| | Polyurethane film prevents allergies |
| | Prevents moisture ingress to reduce infection |
| Ingredients | Polyurethane foam |
| | Polyurethane film |
| | Pressure sensitive adhesive |
| | PP carving paper |

The first processing engine 300 further analyzes the product data of the medical appliances stored in the second database 200 to extract the “featured text information” of the medical appliances. In one example, the first processing engine 300 analyzes the instruction sheet of the medical appliances according to a word segmentation algorithm (e.g., the word segmentation algorithm provided by Academia Sinica in Taiwan, Republic of China), and removes conjunctions (e.g., “or”, “and”, etc.), personal pronouns (e.g., “you”, “I”, “he”, etc.) and punctuation marks, so as to obtain the featured text information.

Moreover, according to different classification attributes, the first processing engine 300 classifies the featured text information. For example, for the dressing D03, the classification attribute of the featured text information “C brand” is: brand. The classification attribute of the featured text information “gel”, “foam”, “polyurethane” and “PP” is: material. The classification attribute of the featured text information “sterilization”, “absorb wound exudate”, “ease for wound observation”, “non-sticky wound”, “prevent allergy”, “prevent water intrusion” and “reduce infection” are: function.

In addition, the first processing engine 300 calculates the occurrence number F_N of the classified featured text information. For example, the total number of all dressings D01˜D300 stored in the second database 200 is, a number of 300. Taking the classification attribute “function” as an example, the occurrence number F_N of the featured text information “sterilization” of dressings D01˜D300 is, a number of 320. The occurrence number F_N of the featured text information “infection” is, a number of 290. The occurrence number F_N of the featured text information “non-sticky” is, a number of 280.

Moreover, the first processing engine 300 may further perform individual statistics for each of the dressings, as shown in Table 2-2 that, the occurrence number F_N of the featured text information “sterilization” of dressing D01 is 38, and the occurrence number F_N of “infection” is 15, and 5 the occurrence number F_N of “non-sticky” is 18. Furthermore, the occurrence number F_N of the featured text information “sterilization” of dressing D02 is 22, and the occurrence number F_N of “infection” is 0, and the occurrence number F_N of “non-sticky” is 62. Moreover, the occurrence number F_N of the featured text information “sterilization” of dressing D02 is 67, and the 10 occurrence number F_N of “infection” is 28, and the occurrence number F_N of “non-sticky” is 0.

TABLE 2-2
Occurrence number F_N of featured text information F_N

featured text information

| medical appliance | | Infection | |
|---|---|---|---|
| (dressing) | Sterilization | Non-stick | Non-stick |
| Dressing D01 | 38 | 15 | 18 |
| skin affinity gel of “A | | | |
| brand”)) | | | |
| Dressing D02 | 22 | 0 | 62 |
| wound powder of “B | | | |
| brand”) | | | |
| Dressing D03 | 67 | 28 | 0 |
| Non-stick wound | | | |
| dressing of “C brand” | | | |

In addition, the first processing engine 300 normalizes the occurrence number F_N of the featured text information, so as to obtain the normalized values shown in Table 2-3.

TABLE 2-3
Normalized value of occurrence number
F_N of featured text information F_N

featured text information

| medical appliance | | Infection | |
|---|---|---|---|
| (dressing) | Sterilization | Non-stick | Non-stick |
| Dressing D01 | 0.25 | 0.1 | 0.12 |
| skin affinity gel of “A | | | |
| brand”)) | | | |
| Dressing D02 | 0.14 | 0 | 0.41 |
| wound powder of “B | | | |
| brand”) | | | |
| Dressing D03 | 0.44 | 0.18 | 0 |
| Non-stick wound | | | |
| dressing of “C brand” | | | |

The first processing engine 300 may set a threshold F_th for the occurrence number F_N of the featured text information. When the featured text information conform to the threshold rule (i.e., the occurrence number F_N of the featured text information is greater than the threshold F_th), the first processing engine 300 determines that the featured text information is “often used featured text information” and serves as “medical appliance keyword”. And, a lexicon 250 is established in the second database 200 to store these “medical appliance keywords”.

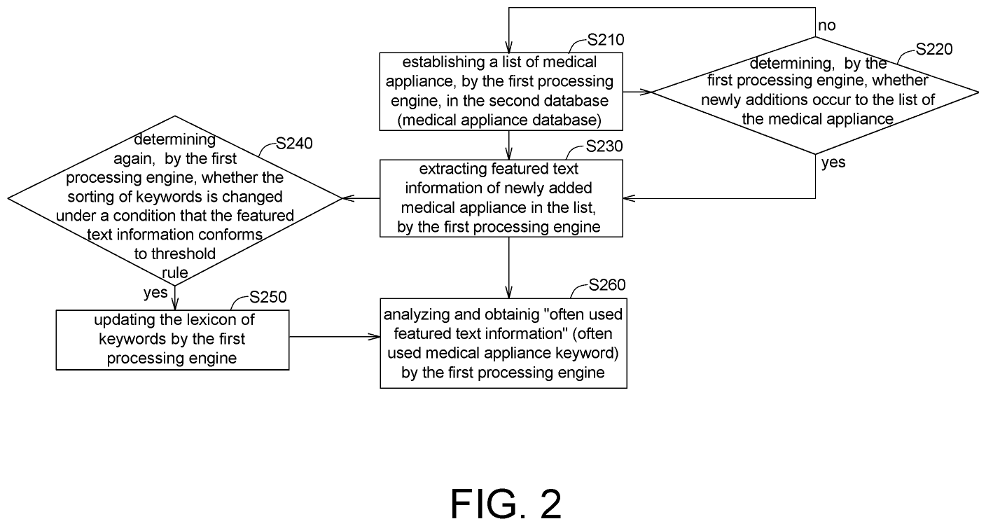

According to the occurrence number F_N of the featured text information, the first processing engine 300 sorts the “medical appliance keywords” in the lexicon 250. Moreover, the first processing engine 300 may perform adaptive updating for the lexicon 250, and the updating scheme is shown as the flow chart in FIG. 2.

Please refer to FIG. 2, which shows a flow chart of adaptive updating for the lexicon 250 of the “medical appliance keyword”, which is performed by the first processing engine 300. First, step S210 is executed: the first processing engine 300 further establishes a list 260 of the medical appliances in the second database 200. For example, the list 260 is, totally 300 kinds of dressings D01˜D300.

Then, step S220 is executed: the first processing engine 300 determines whether newly additions occur to the list 260 of medical appliances. When the list 260 has newly added items, step S230 is executed: for the newly added medical appliance in the list 260, the first processing engine 300 extracts the featured text information.

Then, step S240 is executed: when the featured text information conforms to the threshold rule (i.e., the occurrence number F_N of the featured text information is greater than the threshold F_th), the first processing engine 300 determines again whether the ranking of “medical appliance keywords” has a change. When the ranking of the “medical appliance keywords” changes, step S250 is executed: the first processing engine 300 updates the lexicon 250.

Then, step S260 is executed: the first processing engine 300 analyzes and obtains often used “medical appliance keywords” (i.e., “often used featured text information”).

Please refer to FIG. 1 again, in the data processing system 1000 of the present disclosure, the second processing engine 400 is an analysis engine for “correlation between the nursing records and the medical appliance feature”. The second processing engine 400 is used to obtain the correlation weight value R_SF for the wound state change and the featured text information. The correlation weight value R_SF is referred to as “the second correlation weight value”, which indicates the correlation between the wound state change and the featured text information in the nursing records of wound symptoms.

Taking the wound symptom “granulation” as an example, the wound state x of the “granulation” of cases A001, A002 and A003 in Table 1-3 have normalized values of 0.1, 0.5 and 0.3, respectively. On the other hand, usage frequencies of 0.1, 0.1 and 0.2 for the dressing D01, dressing D02 and dressing D03 correspond to the cases A001, A002 and A003 in Tables 1-4. Accordingly, the second processing engine 400 analyzes and obtains that, the correlation weight value R_SD of the wound symptom “granulation” and the dressing D01 is 0.36, the correlation weight value R_SD of the wound symptom “granulation” and the dressing D02 is 0.18, and the correlation weight value R_SD of the wound symptom “granulation” and the dressing D03 is 0.12.

The correlation weight value R_SD of wound symptom (or wound state) and medical appliances (e.g., dressings) is referred to as “first correlation weight value”, which indicates the correlation between wound symptoms (or wound state) and medical appliances.

Similarly, taking the wound symptom “necrosis” as an example, the normalized values of the wound state x of “necrosis” for cases A001, A002 and A003 in Table 1-3 are 0.45, 0.2 and 0.5 respectively. On the other hand, usage frequencies of 0.1, 0.1 and 0.2 for the dressing D01, dressing D02 and dressing D03 correspond to the cases A001, A002 and A003 in Tables 1-4. Accordingly, the second processing engine 400 analyzes and obtains: the correlation weight value R_SD of wound symptom “necrosis” and the dressing D01 is 0.31. The correlation weight value R_SD of wound symptom “necrosis” and the dressing D02 is 0.24. The correlation weight value R_SD of wound symptom “necrosis” and the dressing D03 is 0.15.

From the above, according to the normalized value of the wound state x in Table 1-3 and the normalized value of the treatment record in Table 1-4 (such as, the usage frequency of medical appliances), the second processing engine 400 may analyze and obtain the correlation weight value R_SD of wound symptom and medical appliances (dressings) shown in Table 3-1.

TABLE 3-1
Correlation weight value R_SD between
wound symptom and medical appliance

| | Medical appliance (dressing) | | |
|---|---|---|---|
| | D01 | D02 | D03 |
| Wound | Product name | Product name | Product name |
| symptom | a1 | a2 | a3 |
| long (unit: mm) | 0.24 | 0.18 | 0 |
| width (unit: mm) | 0.48 | 0.04 | 0.4 |
| Granulation | 0.36 | 0.18 | 0.12 |
| Necrosis | 0.31 | 0.24 | 0.15 |

In addition, according to the correlation weight value R_SD of the wound symptom and medical appliances (dressings) in Table 3-1 and the normalized value of the occurrence number F_N of the featured text information in Table 2-3, the second processing engine 400 analyzes the correlation between the state change of wound symptoms and the featured text information of medical appliances, so as to obtain the correlation weight value R_SF of the wound state change and featured text information in Table 3-2.

For example, in Table 3-1, the correlation weight values R_SD of the wound symptom “granulation” and dressing D01, dressing D02 and dressing D03 are, 0.36, 0.18 and 0.12 respectively. In Table 2-3, the normalized values of the occurrence number F_N of the featured text information “sterilization” for dressing D01, dressing D02 and dressing D03 are, 0.25, 0.14 and 0.44 respectively. The correlation weight value R_SD in Table 3-1 and the normalized values of the occurrence number F_N in Table 2-3 are applied to the calculation of formula (3), and the second processing engine 400 calculates that, the correlation weight value R_SF of the wound state change of wound symptom “granulation” and the featured text information “sterilization” is 0.138.

$$\begin{matrix}R\_SF ( Granulation / Sterilization ) = \{ R\_SD ( Granulation / \\ Dressing D 01 ) \times \\ F\_N ( Dressing D 01 / \\ Sterilizationa ) + \\ R\_SD ( Granulation / \\ Dressing D 01 ) \times \\ F\_N ( Dressing D 01 / \\ Sterilizationa ) + \\ R\_SD ( Granulation / \\ Dressing D 03 ) \times \\ F\_N ( Dressing D 03 / \\ Sterilizationa ) \} \div 3 \\ = \{ ( 0.36 \times 0.25 ) + \\ ( 0.18 \times 0.14 ) + \\ ( 0.12 \times 0.44 ) \} \div 3 \\ = 0.138\end{matrix} \qquad formula ( 3 )$$

Similarly, according to the calculation of formula (4), the second processing engine 400 calculates that, the correlation weight value R_SF of the wound state change of the wound symptom “granulation” and the featured text information “infection” is 0.06.

$$\begin{matrix}R\_SF ( Granulation / infection ) = \{ R\_SD ( Granulation / \\ Dressing D 01 ) \times \\ F\_N ( Dressing D 01 / \\ Infection ) + \\ R\_SD ( Granulation / \\ Dressing D 02 ) \times \\ F\_N ( Dressing D 02 / \\ Infection ) + \\ R\_SD ( Granulation / \\ Dressing D 03 ) \times \\ F\_N ( Dressing D 03 / \\ Infection ) \} \div 3 \\ = \{ ( 0.36 \times 0.1 ) + ( 0.18 \times 0 ) + \\ ( 0.12 \times 0.18 ) \} \div 3 \\ = 0.06\end{matrix} \qquad formula ( 4 )$$

TABLE 3-2
Correlation weight value R_SF between wound
state changing (of wound symptom) and featured
text information (of medical appliance)

| | featured text information | | |
|---|---|---|---|
| wound symptom | Sterilization | Infection | Non-stick |
| Long | 0.066 | 0.024 | 0.096 |
| width | 0.26 | 0.128 | 0.064 |
| Granulation | 0.138 | 0.06 | 0.108 |
| necrosis | 0.146 | 0.061 | 0.127 |

Please refer to FIG. 1 again, in the data processing system 1000 of the present disclosure, the input unit 600 is used for inputting that, the target state y related to the target nursing scheme. For example, if the user (nursing staff) expects the target state y of the wound to be “50%” for the tissue growth area, then the target state y inputted by the user through the input unit 600 is “flesh growth of 50%”. For another example, if the target state y of the medical appliances which the user expects to treat the wounds is, reducing the cost down to 30%, then the target state y inputted by the user through the input unit 600 is “cost of 30%”.

The third processing engine 500 of the data processing system 1000 is an analysis and recommendation engine of “keywords of target nursing scheme”. The third processing engine 500 analyzes the target state y of the wound, and fetches the field corresponding to the wound symptom state from the wound symptom record which is stored in the first database 100. Moreover, the third processing engine 500 selects the “query condition” according to the target state y and the trend Δx of the wound state. For example, the inputted target state y is “flesh growth of 50%”, and the third processing engine 500 analyzes that the query condition is, the granulation area >50%.

According to the selected query condition, the third processing engine 500 analyzes the keywords of the target nursing schemes (also referred to as “target nursing keywords”). According to the “target nursing keywords”, the third processing engine 500 recommends the most suitable medical appliances for wound treatment. That is, according to the “target nursing keywords”, the third processing engine 500 selects a suitable “recommended medical appliance” from the medical appliances recorded in the second database 200.

For example, the target state y is “flesh growth of 50%”, the query condition is “granulation area >50%”, and the “target nursing keyword” analyzed by the third processing engine 500 is “granulation growth”. Then, according to the “target nursing keyword”, the third processing engine 500 calculates the scores of each of the medical appliances in the second database 200. For example, for the “target nursing keyword” being “granulation growth”, the featured text information “sterilization”, “non-sticky” and “skin affinity” have higher value of correlation weight value R_SF for the wound symptom “granulation”. Then, scores of each of the medical appliances are calculated for the “sterilization”, “non-stick” and “skin affinity”.

Similar to the normalized value of the occurrence number F_N of the featured text information of each medical appliance in Table 2-3, Table 4-1 lists more medical appliances and featured text information.

TABLE 4-1
Normalized value of occurrence number F_N of featured text information

| | Good skin | Hydrophilic | Sterilization | | Non-sticking | silicone | |
|---|---|---|---|---|---|---|---|
| | affinity | (V) | (V) | powder | (V) | fraction | score |
| Dressing D01 | 0.8 | 0.4 | 0.25 | 0.9 | 0.12 | 0 | 1.17 |
| Dressing D02 | 0.5 | 0.6 | 0.14 | 0 | 0.41 | 0.4 | 1.05 |
| Dressing D03 | 0.7 | 0.7 | 0.44 | 0 | 0 | 0.5 | 1.14 |
| Dressing D04 | 0.8 | 0.8 | 0.8 | 0 | 0.8 | 0.8 | 2.4 |
| Dressing D05 | 0.3 | 0.6 | 0.4 | 0 | 0.6 | 0.8 | 1.3 |
| Dressing D06 | 0.7 | 0.8 | 0.8 | 0.9 | 0.8 | 0.2 | 2.3 |
| Dressing D07 | 0.4 | 0.8 | 0.6 | 0 | 0.7 | 0.8 | 1.7 |
| Dressing D08 | 0.9 | 0.8 | 0.8 | 0 | 0.7 | 0.2 | 2.4 |
| . . . | | | | | | | |

In Table 4-1, the normalized values of the occurrence number F_N corresponding to the featured text information “skin affinity”, “sterilization” and “non-sticky” of dressing D01 are 0.8, 0.25 and 0.12 respectively. Adding the above normalized values, a score of 1.17 for Dressing D01 is obtained. Similarly, the normalized values of the occurrence number F_N of the featured text information “ skin affinity”, “sterilized” and “non-sticky” of dressing D02 are 0.5, 0.14 and 0.41 respectively. Adding the above normalized values, a score of 1.05 for dressing D02 is obtained. The normalized values of the occurrence number F_N of the featured text information “skin affinity”, “sterilization” and “non-sticky” of dressing D03 are 0.7, 0.44 and 0, respectively. Adding the above normalized values, a score of 1.14 for dressing D03 is obtained. Likewise, the third processing engine 500 calculates the scores of other dressings D04˜D08 as 2.4, 1.3, 2.3, 1.7 and 2.4.

Moreover, according to the scores of all dressings in the second database 200, the third processing engine 500 sorts all dressings. According to the sorting of dressings, the third processing engine 500 recommends the most suitable dressings as “recommended medical appliances” which are suitable for treating wounds. Table 4-2 shows the sorting of the 8 dressings D01˜D08 in Table 4-1, and the third processing engine 500 recommends dressing 04, dressing 08 and dressing 06 with the highest ranking. These dressing 04, dressing 08 and dressing 06 are “recommended medical appliances” suitable for treating wounds.

TABLE 4-2
Normalized value of occurrence number F_N of featured text information

| | Good skin | Hydrophilic | Sterilization | | Non-sticking | silicone | |
|---|---|---|---|---|---|---|---|
| | affinity | (V) | (V) | powder | (V) | fraction | score |
| Dressing D04 | 0.8 | 0.8 | 0.8 | 0 | 0.8 | 0.8 | 2.4 |
| Dressing D08 | 0.9 | 0.8 | 0.8 | 0 | 0.7 | 0.2 | 2.4 |
| Dressing D06 | 0.7 | 0.8 | 0.8 | 0.9 | 0.8 | 0.2 | 2.3 |
| Dressing D07 | 0.4 | 0.8 | 0.6 | 0 | 0.7 | 0.8 | 1.7 |
| Dressing D05 | 0.3 | 0.6 | 0.4 | 0 | 0.6 | 0.8 | 1.3 |
| Dressing D01 | 0.8 | 0.4 | 0.25 | 0.9 | 0.12 | 0 | 1.17 |
| Dressing D03 | 0.7 | 0.7 | 0.44 | 0 | 0 | 0.5 | 1.14 |
| Dressing D02 | 0.5 | 0.6 | 0.14 | 0 | 0.41 | 0.4 | 1.05 |

Please refer to FIG. 1 again, the output unit 700 of the data processing system 1000 outputs “recommended medical appliances” to the user. For example, the output unit 700 outputs dressing 04, dressing 08 and dressing 06 with the highest ranking, and their product names are respectively: “non-stick wound dressing of “D brand””, “skin affinity gel of “H brand”” and “silicone gel foam of “F brand””.

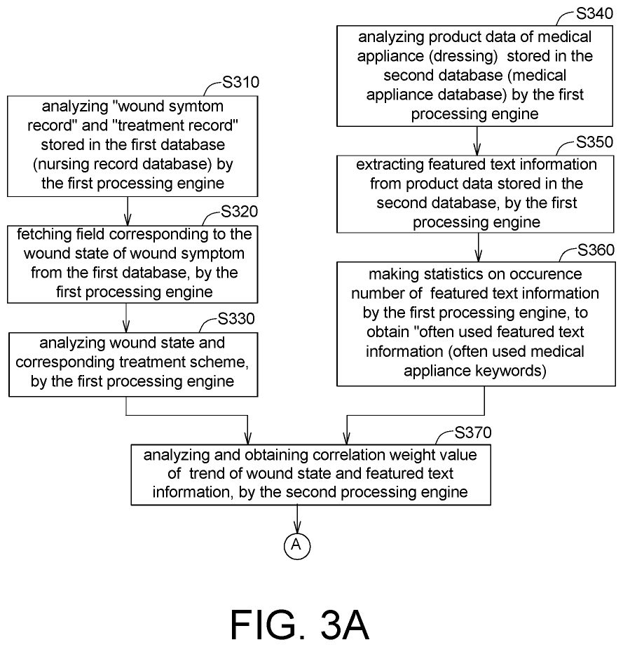

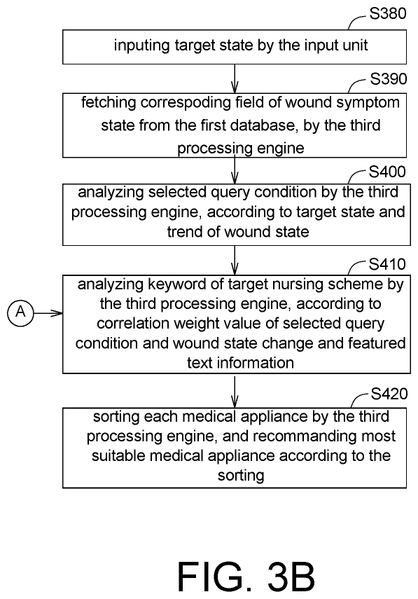

Please refer to FIGS. 3A and 3B, which are flowcharts of a data processing method according to an embodiment of the present disclosure. The data processing methods in FIGS. 3A and 3B may be executed by the data processing system 1000 in FIG. 1. As shown in FIG. 3A, firstly, step S310 is executed: the first database 100 is analyzed by the first processing engine 300, and the first database 100 is a “nursing record database”. Comprising: analyzing the “wound symptom record” shown in Table 1-1 and the “treatment record” shown in Table 1-2.

Then, step S320 is executed: the first processing engine 300 fetches the field corresponding to the wound state of the wound syndrome from the first database 100.

Then, step S330 is executed: analyzing the wound state and the corresponding treatment scheme by the first processing engine 300.

Then, step S340 is executed: analyzing the second database 200 by the first processing engine 300, and the second database 200 is a “medical appliance database”. For example, the first processing engine 300 analyzes various product data of the medical appliances (such as dressings), comprising: the product name of the dressing, various indications, various product features and various ingredients, which are shown in Table 2-1.

Then, step S350 is executed: according to the various product data of the medical appliances stored in the second database 200, the featured text information is extracted by the first processing engine 300. Moreover, the extracted featured text information is classified according to classification attributes, such as brand, material, function, and so on.

Then, step S360 is executed: calculating the occurrence number F_N of the classified featured text information by the first processing engine 300, and obtaining “often used featured text information” from the featured text information according to the threshold rule. The often used featured text information is taken as often used “medical appliance keywords”.

Then, step S370 is executed: according to the treatment scheme of the medical appliances corresponding to the wound symptoms obtained in step S330 and the “often used featured text information” obtained in step S360, the second processing engine 400 analyzes the correlation between the state changes of the wound symptoms and the featured text information of medical appliances, so as to obtain the correlation weight value R_SF of the wound state change and featured text information in Table 3-2.

Then, in FIG. 3B, step S380 is executed: inputting the target state y related to the target nursing scheme, through the input unit 600.

Then, step S390 is executed: analyzing the target state y by the third processing engine 500, and extracting the field corresponding to the wound symptom state from the wound symptom record stored in the first database 100.

Then, step S400 is executed: according to the target state y and the trend Δx of the wound state in the first database 100, analyzing and selecting the “query condition” by the third processing engine 500.

Then, step S410 is executed: according to the query condition obtained in step S400 and the correlation weight value R_SF for the wound state change and featured text information obtained in step S370, analyzing the “target nursing keyword” by the third processing engine 500.

Then, step S420 is executed: according to the “target nursing keyword”, the third processing engine 500 calculates the scores of each medical appliance in the second database 200, sorts each medical appliance according to the score, and selects “recommended medical appliances” most suitable for treating wounds according to the ranking.

It will be apparent to those skilled in the art that various modifications and variations may be made to the disclosed embodiments. It is intended that the specification and examples be considered as exemplary only, with a true scope of the disclosure being indicated by the following claims and their equivalents.

What is claimed is:

1. A data processing system, comprising:
a first database, for storing a wound symptom record and a treatment record, the wound symptom record comprises a plurality of wound states, and the treatment record comprises a plurality of medical appliances corresponding to the wound states;
a second database, for storing a plurality of product data of the medical appliances;
a first processing engine, for analyzing the first database to analyze a trend of each of the wound states according to the wound symptom record, and analyzing the second database to extract a plurality of featured text information from the product data;
a second processing engine, for generating a plurality of first correlation weight values and a plurality of second correlation weight values of the wound states, the first correlation weight values indicate correlations between the wound states and the medical appliances, the second correlation weight values indicate correlations between the wound states and the featured text information; and
a third processing engine, for generating a query condition according to a target state, generating a target nursing keyword according to the query condition, and selecting a plurality of recommended medical appliances from the medical appliances according to the target nursing keyword and the second correlation weight values,
wherein, the query condition is related to the trend of each of the wound states, and the target nursing keyword is related to the target state.

2. The data processing system according to claim 1, further comprising:
an input unit, for inputting the target state, the target state is related to a target nursing scheme of the wound states;
wherein, the third processing engine fetches fields corresponding to the wound states from the wound symptom record in the first database according to the target state.

3. The data processing system according to claim 2, further comprising:
an output unit, for outputting product names of the recommended medical appliances;
wherein, the recommended medical appliances conform to the target nursing scheme and the query condition.

4. The data processing system according to claim 1, wherein:
the first processing engine analyzes a normalized value of each of the wound states according to the wound symptom record, and calculates a usage frequency of the medical appliances from the treatment record; and
the second processing engine obtains the first correlation weight values according to the normalized value of each of the wound states and the usage frequency of the medical appliances.

5. The data processing system according to claim 1, wherein:
the first processing engine calculates an occurrence number of the featured text information of the medical appliances; and
the second processing engine obtains the second correlation weight values according to the first correlation weight values and the occurrence number of the corresponding featured text information of the medical appliances.

6. The data processing system according to claim 5, wherein, the third processing engine analyzes the second correlation weight values of the wound states corresponding to the target nursing keyword, and selects the featured text information with higher values corresponding to the second correlation weight values, calculating scores of the medical appliances according to the occurrence number of the featured text information, and selects the medical appliances with higher scores as the recommended medical appliances.

7. The data processing system according to claim 5, wherein, when the occurrence number of the featured text information is greater than a threshold, the featured text information are used as a plurality of medical appliance keywords.

8. The data processing system according to claim 7, wherein, the second database further comprising:
a lexicon, for storing the medical appliance keywords;
wherein, when the medical appliances in the second database comprise a new medical appliance, the first processing engine extracts a plurality of new featured text information of the new medical appliances and updates the lexicon.

9. The data processing system according to claim 1, wherein, the medical appliances are a plurality of dressings, and the first processing engine extracts the featured text information from an instruction sheet of each of the dressings.

10. The data processing system according to claim 1, wherein, the first processing engine classifies the featured text information according to a plurality of classification attributes, and the classification attributes at least comprise brands, materials and functions of the medical appliances.

11. A data processing method, comprising:
storing a wound symptom record and a treatment record in a first database, the wound symptom record comprises a plurality of wound states, and the treatment record comprises a plurality of medical appliances corresponding to the wound states;
storing a plurality of product data of the medical appliances in a second database;
analyzing the first database to analyze a trend of each of the wound states according to the wound symptom record and analyzing the second database to extract a plurality of featured text information from the product data, through a first processing engine;
generating a plurality of first correlation weight values and a plurality of second correlation weight values of the wound states through a second processing engine, the first correlation weight values indicate correlations between the wound states and the medical appliances, the second correlation weight values indicate correlations between the wound states and the featured text information; and
generating a query condition according to a target state, generating a target nursing keyword according to the query condition, and selecting a plurality of recommended medical appliances from the medical appliances according to the target nursing keyword and the second correlation weight values, through a third processing engine,
wherein, the query condition is related to the trend of each of the wound states, and the target nursing keyword is related to the target state.

12. The data processing method according to claim 11, further comprising:
inputting the target state through an input unit, the target state is related to a target nursing scheme of the wound states;
wherein, fetching fields corresponding to the wound states from the wound symptom record in the first database according to the target state, through the third processing engine.

13. The data processing method according to claim 12, further comprising:
outputting product names of the recommended medical appliances through an output unit;
wherein, the recommended medical appliances conform to the target nursing scheme and the query condition.

14. The data processing method according to claim 11, wherein:
analyzing a normalized value of each of the wound states according to the wound symptom record, and calculating a usage frequency of the medical appliances from the treatment record, through the first processing engine; and
obtaining the first correlation weight values according to the normalized value of each of the wound states and the usage frequency of the medical appliances, through the second processing engine.

15. The data processing method according to claim 11, wherein:
calculating an occurrence number of the featured text information of the medical appliances, through the first processing engine; and
obtaining the second correlation weight values according to the first correlation weight values and the occurrence number of the corresponding featured text information of the medical appliances, through the second processing engine.

16. The data processing method according to claim 15, wherein, analyzing the second correlation weight values of the wound states corresponding to the target nursing keyword, and selecting the featured text information with higher values corresponding to the second correlation weight values, calculating scores of the medical appliances according to the occurrence number of the featured text information, and selecting the medical appliances with higher scores as the recommended medical appliances, through the third processing engine.

17. The data processing method according to claim 15, wherein, when the occurrence number of the featured text information is greater than a threshold, the featured text information are used as a plurality of medical appliance keywords.

18. The data processing method according to claim 17, further comprising:
establishing a lexicon in the second database; and
storing the medical appliance keywords in the lexicon,
wherein, when the medical appliances in the second database comprise a new medical appliance, the first processing engine extracts a plurality of new featured text information of the new medical appliances and updates the lexicon.

19. The data processing method according to claim 11, wherein, the medical appliances are a plurality of dressings, and extracting the featured text information from an instruction sheet of each of the dressings through the first processing engine.

20. The data processing method according to claim 11, wherein, classifying the featured text information according to a plurality of classification attributes through the first processing engine, and the classification attributes at least comprise brands, materials and functions of the medical appliances.
